(19) United States
(12) Patent Application Publication
(10) Pub. No.: US 2026/0256758 A1
(43) Pub. Date: Sep. 3, 2026

(54) COMPOUND AND PHARMACEUTICAL COMPOSITION COMPRISING THE SAME FOR TREATING TUBERCULOSIS INFECTION

(71) Applicants: KOREA ATOMIC ENERGY RESEARCH INSTITUTE, Daejeon (KR); KYUNGPOOK NATIONAL UNIVERSITY INDUSTRY-ACADEMIC COOPERATION FOUNDATION, Daegu (KR)

(72) Inventors: Ha Yeon SONG, Daejeon (KR); Eui Baek BYUN, Daejeon (KR); Bo Gyeong YOO, Daejeon (KR); Jong Ho JEON, Daeju (KR)

(73) Assignees: KOREA ATOMIC ENERGY RESEARCH INSTITUTE, Daejeon (KR); KYUNGPOOK NATIONAL UNIVERSITY INDUSTRY-ACADEMIC COOPERATION FOUNDATION, Daegu (KR)

(21) Appl. No.: 19/552,351

(22) Filed: Feb. 27, 2026

(30) Foreign Application Priority Data
Feb. 28, 2025 (KR) 10-2025-0026843
Feb. 26, 2026 (KR) 10-2026-0036026

(51) Int. Cl. A61K 31/444; A61K 31/4409; A61K 31/4709; A61K 31/497; A61K 31/498; A61P 11/00

(52) U.S. Cl. CPC A61K 31/444; A61K 31/4409; A61K 31/4709; A61K 31/497; A61K 31/498; A61P 11/00

(57) ABSTRACT

The present disclosure relates to a compound represented by Chemical Formula 1 or a pharmaceutically acceptable salt thereof, for treating tuberculosis infection; and a pharmaceutical composition for treating tuberculosis infection comprising the same, and more particularly, to a pharmaceutical composition for the treatment of a disease caused by Mycobacterium tuberculosis infection comprising as an active ingredient a compound represented by the following Chemical Formula 1 or a pharmaceutically acceptable salt thereof.

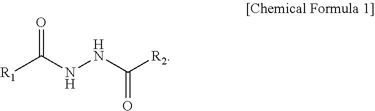

In Chemical Formula 1, R₁ and R₂ are independently selected from the group consisting of a nitrogen-containing C₃-C₉ heteroaryl group and a C₆-C₁₂ aryl group.

### CROSS-REFERENCE TO RELATED APPLICATIONS

This application claims benefit of priority under 35 U.S.C. § 119(a) to Korean patent application number 10-2025-0026843, filed on Feb. 28, 2025, and Korean patent application number 10-2026-0036026, filed on Feb. 26, 2026, in the Korean Intellectual Property Office, the disclosure of which is incorporated herein by reference in its entirety.

### DETAILED DESCRIPTION

### Technical Field

The present disclosure relates to a compound for treating tuberculosis infection and a pharmaceutical composition comprising the same, and more particularly to a compound having a novel structure useful for treating tuberculosis infections and to a pharmaceutical composition comprising the same as active ingredients, which may not only be effective in treating tuberculosis but also in treating granulomas, a major pathological condition associated with tuberculosis.

### BACKGROUND

Tuberculosis (TB), which has a long history, may remain one of the top ten causes of death, and as the principle of treatment thereof, the initial therapy may be a six-month short-course regimen. Generally, a four-drug combination of first-line anti-tuberculosis agents rifampin (RIF), isoniazid (INH), pyrazinamide (PZA), and ethambutol (EMB) may be used, and after two months, the three drugs RIF, INH, and EMB may be continued for four months. Streptomycin (STR) may be used instead of EMB during the initial two-month intensive phase. In that case, the continuation phase may consist of administration of RIF and INH.

Retreatment for patients with relapse typically may involve resuming the original treatment, as most patients remain susceptible to the initial treatment, and treatment may be extended by three months beyond the initial treatment period. For patients who failed the initial treatment, re-treatment may involve performing drug susceptibility testing to exclude all previously administered drugs and administering a combination of at least three, and preferably four or more, new, previously unused susceptibility drugs. Treatment may be generally continued for at least 18 months.

The biggest problem of tuberculosis treatment is that no effective specialized therapy is present when resistance to first-line anti-tuberculosis drugs develops. As the development of effective anti-tuberculosis drugs and systematic management have dramatically reduced the incidence and mortality of tuberculosis, the emergence of multidrug-resistant tuberculosis (MDR-TB) and the spread of acquired immunodeficiency syndrome (AIDS) have recently contributed to an increase in tuberculosis. Recently, drug-resistant tuberculosis, including multidrug-resistant tuberculosis, has emerged as a serious public health problem. In recent years, the tuberculosis infection rate in Korea has not continued to decline, which is closely related to the fact that the number of patients with drug-resistant tuberculosis has not decreased. In addition, it is reported that 50% of patients with drug-resistant tuberculosis die. Therefore, a substantial proportion of tuberculosis-related deaths are attributable to drug-resistant tuberculosis. Although several second-line anti-tuberculosis drugs have been developed, none can replace first-line drugs in terms of efficacy. Therefore, the development of new anti-tuberculosis agents with mechanisms of action different from existing drugs is an urgent task.

Meanwhile, granulomas, the major pathological lesions associated with tuberculosis (TB), are dense aggregates of cell debris, bacteria, and host immune cells that surround macrophages infected with Mycobacterium tuberculosis (Mtb). The primary cause of granuloma formation is mycolic acids in the Mtb cell wall, which inhibit phagosome maturation and phagolysosome fusion and promote inflammatory responses. Granulomas persist in all stages of tuberculosis, including active, latent, and recurrent disease, and, in particular, in latent tuberculosis, Mtb enters a dormant state under nutrient deprivation and hypoxic conditions while still maintaining metabolic activity. Such granulomas are one of the main causes of reduced success rates in tuberculosis treatment. Meanwhile, the first-line anti-tuberculosis drug isoniazid is ineffective against dormant Mtb within granulomas, which can decrease therapeutic effect and promote the development of resistance. In addition, sub-dose delivery of drugs to bacteria inside granulomas can impair therapeutic effect and induce resistance. Because of these problems, a strategy known as host-directed therapy (HDT), which treats disease by modulating host immune responses or physiological pathways rather than directly targeting the pathogen, has recently attracted attention in tuberculosis treatment.

### SUMMARY

An aspect of the present disclosure is to provide compounds having new structures that are useful for treating tuberculosis infection.

According to an aspect of the present disclosure is to provide a pharmaceutical composition comprising, as an active ingredient, a compound having a novel structure that is useful for treating tuberculosis infection and that is effective not only for tuberculosis but also for treating granulomas, a major pathological condition associated with tuberculosis.

According to an aspect of the present disclosure, there is provided a compound represented by Chemical Formula 1 below, or a pharmaceutically acceptable salt thereof, for the treatment of tuberculosis infection.

According to another aspect of the present disclosure, a pharmaceutical composition for treating Mycobacterium tuberculosis infection is provided, comprising, as an active ingredient, a compound represented by the following Chemical Formula 1 or a pharmaceutically acceptable salt thereof.

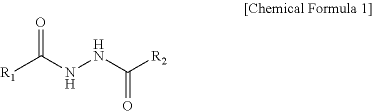

In Chemical Formula 1, R₁ and R₂ are independently selected from the group consisting of a nitrogen-containing C₃-C₉ heteroaryl group and a C₆-C₁₂ aryl group.

### Advantageous Effects

According to the present disclosure, a novel compound for treating tuberculosis infection is provided that effectively inhibits inflammatory mediators and, by alleviating hypoxic conditions occurring during infiltration of immune cells into tissues, effectively ameliorates granulomatous lesions, and compositions comprising the compound of the present disclosure are expected, from a host-directed therapy (HDT) perspective during tuberculosis treatment, to be useful as therapeutics or adjuvants for the treatment, alleviation, and improvement of tuberculosis and granulomatous lesions.

### BRIEF DESCRIPTION OF THE DRAWINGS

FIG. 1 shows peaks resulting from LC-MS analysis of isoniazid irradiated with gamma rays at 50 kGy.

FIG. 2 illustrates compounds corresponding to each peak identified in the LC-MS analysis of FIG. 1.

FIG. 3 illustrates the results of evaluating cell viability after treatment with varying concentrations of isoniazid (INH) and derivatives INH-D1 to INH-D5 to assess cytotoxicity.

FIG. 4 is a graph illustrating the production of the inflammatory cytokines IL-6 and TNF-α as a function of the concentrations of INH and INH derivatives (INH-D1 to INH-D5).

FIG. 5 illustrates the effect of INH and INH derivatives on the expression of CD80 and CD86 costimulatory molecules induced by TDM stimulation.

FIG. 6 illustrates the effect of INH and INH derivatives on TDM-stimulated expression of MHC class I and MHC class II.

FIG. 7 is a graph showing the inhibitory effects on production of the inflammatory cytokines IL-6 and TNF-α at different concentrations of INH derivatives (INH-D1 to INH-D10), INH, and Dexa.

FIG. 8 is a schematic diagram illustrating the administration and sacrifice regimen of an animal study using a mouse model to assess whether pulmonary granulomatous lesions are alleviated.

FIG. 9 is a graph illustrating the body weights of mice measured over 7 days for the lung granuloma induction group (TDM only) and for groups administered INH derivatives (INH-D1 and INH-D2).

FIG. 10 shows photographs of lungs excised from mice sacrificed on day 7 for the pulmonary granuloma induction group (TDM only) and for groups administered INH derivatives (INH-D1 and INH-D2) (FIG. 10 (left)), and a graph of the lung index (lung weight/body weight×100) calculated after weighing (FIG. 10 (right)).

FIG. 11 shows photographs (FIG. 11 (left)) of tissue lesions observed following histopathological analysis after sacrificing mice and extracting lungs on day 7 for the pulmonary granuloma induction group (TDM only) and the group administered INH derivatives, and a graph (FIG. 11 (right)) showing the area occupied by granuloma lesions in the lungs.

FIGS. 12A-12D are graphs showing, as a result of verifying the cellular composition of lung tissue using a flow cytometer, the total number of white blood cells (CD45+ cells) (FIG. 12A), the improvement in hypoxic conditions (FIG. 12B), the number of interstitial macrophages (FIG. 12C), and the number of dendritic cells (FIG. 12D).

FIG. 13A and FIG. 13B show results of analysis of cytokines and chemokines in serum, in which the cytokine IFN-γ, the neutrophil-infiltrating chemokine KC, the macrophage/monocyte-infiltrating chemokine MCP-1, RANTES inducing T cell and monocyte infiltration, IL-6, and TNF-α were detected on day 3 (FIG. 13A) and on day 5 (FIG. 13B) after pulmonary granuloma induction, respectively.

FIG. 14 shows photographs of lungs excised from mice sacrificed on day 7 in the pulmonary granuloma induction group (TDM only) and for groups administered INH derivative (INH-D2, INH-D4, INH-D6, and INH-D8) (FIG. 14 (left)), and a graph showing the lung index (lung weight/body weight×100) calculated after weighing (FIG. 14 (right)).

FIG. 15 shows photographs (FIG. 15 (top)) of tissue lesions observed following histopathological analysis of lungs extracted from mice sacrificed on day 7 in the lung granuloma induction group (TDM only) and for groups administered INH derivatives (INH-D2, INH-D4, INH-D6, and INH-D8), and a graph (FIG. 15 (bottom)) showing the area occupied by granuloma lesions in the lungs.

### DETAILED DESCRIPTION FOR CARRYING OUT THE DISCLOSURE

Hereinafter, preferred embodiments of the present disclosure will be described with reference to the accompanying drawings. However, the embodiments of the present disclosure may be modified into various other forms, and the scope of the present disclosure is not limited to the embodiments described below.

The present disclosure provides compounds and pharmaceutical compositions comprising the same for effectively controlling tuberculosis infection disease. Here, the term tuberculosis infection includes not only tuberculosis caused by Mycobacterium tuberculosis, but also diseases or symptoms, such as granulomas, that are induced by or associated with tuberculosis.

In the present disclosure, when the term ‘treatment’ is used, it is to be understood as referring to a pharmaceutical composition that may involve not only treatment but also alleviation and/or improvement effects. As used herein, “improvement,” “alleviation” or “treatment” means any action by which the symptoms of the disease are ameliorated or otherwise beneficially altered by administration of the composition.

In the present disclosure, ‘Cx-Cy’ means that the substituent contains X to Y number of carbon atoms. For example, ‘C₆-C₁₂’ means that the substituent contains 6 to 12 carbon atoms, and the range ‘X to Y’ includes all values between X and Y, including X and Y. According to the present disclosure, a novel isoniazid derivative compound is provided, and the novel isoniazid derivative compound of the present disclosure may be used to treat tuberculosis.

More specifically, the compound of the present disclosure is a compound represented by Chemical Formula 1, or a pharmaceutically acceptable salt thereof, for treating tuberculosis infection.

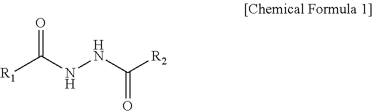

In Chemical Formula 1, R₁ and R₂ are independently selected from the group consisting of a nitrogen-containing C₃-C₉ heteroaryl group and a C₆-C₁₂ aryl group.

The salt of the isoniazid derivative compound of the present disclosure may be in the form of a pharmaceutically acceptable salt, and the term “pharmaceutically acceptable salt” as used herein refers to a form of the compound that does not cause serious irritation to an organism to which the compound is administered and that does not impair the biological activity or physical properties of the compound. The pharmaceutically acceptable salts include acids that form non-toxic acid addition salts containing pharmaceutically acceptable anions, for example, inorganic acids such as hydrochloric acid, sulfuric acid, nitric acid, phosphoric acid, hydrobromic acid, hydroiodic acid (iodic acid), perchloric acid, and stannic acid, and organic carboxylic acids such as succinic acid, oxalic acid, tartaric acid, mandelic acid, propionic acid, citric acid, lactic acid, glycolic acid, gluconic acid, formic acid, acetic acid, trichloroacetic acid, trifluoroacetic acid, benzoic acid, fumaric acid, maleic acid, and salicylic acid, and sulfonic acids such as methanesulfonic acid, ethanesulfonic acid, benzenesulfonic acid, p-toluenesulfonic acid, and naphthalenesulfonic acid. The compounds according to the present disclosure may be converted to their salts by conventional methods.

The compound of Chemical Formula 1 of the present disclosure may be synthesized by any synthetic process well known in the art, and the synthetic steps are not limited. For example, it may also be obtained by electron-beam irradiation.

In the present disclosure, “aryl” refers to an organic radical derived from an aromatic hydrocarbon by removal of one hydrogen atom, and includes a single-ring or fused-ring system. For example, the aryl group includes substituent, but is not limited to, phenyl, naphthyl, biphenyl, anthryl, fluorenyl, phenanthryl, triphenylenyl, pyrenyl, perylenyl, chrysenyl, naphthacenyl, and fluoranthenyl. “Heteroaryl” refers to an aromatic ring derivative having a heterocycle and formed by removal of one or more hydrogen atoms from the ring to yield an aromatic radical, wherein the heteroatom(s) may be one or more selected from the group consisting of S, N, O, Si, P, and B. Specific examples include pyridinyl, pyrimidinyl, pyrazinyl, and the like.

For example, in the compound of Chemical Formula 1 of the present disclosure, a nitrogen-containing C₃-C₉ heteroaryl group may be selected from pyrrolyl, pyrazolyl, imidazolyl, triazolyl, pyridyl, pyridazinyl, pyrimidinyl, pyrazinyl, indolyl, quinolyl, isoquinolyl, cinnolinyl, phthalazinyl, quinazolinyl, quinoxalinyl, and naphthyridinyl.

For example, in a compound of Chemical Formula 1 of the present disclosure, the C₆-C₁₂ aryl group may be selected from phenyl, naphthyl, and biphenylyl.

The compound of Chemical Formula 1 of the present disclosure may, for example, have R₁ selected from the group consisting of pyridyl, pyridazinyl, pyrimidinyl, and pyrazinyl, and R₂ selected from the group consisting of phenyl, pyridyl, pyridazinyl, pyrimidinyl, pyrazinyl, quinolyl, quinoxalinyl, and naphthyl and preferably, R₁ is pyridine, and R₂ is selected from the group consisting of pyridine, phenyl, pyrazinyl, quinolyl, quinoxalinyl, and naphthyl.

For example, in the compound of Chemical Formula 1, when R₁ and R₂ are each pyridyl, the R₁ pyridine may be bonded to the backbone at its 2- or 4-position, and the R₂ pyridine may be bonded to the backbone at its 3- or 4-position.

More specifically, the compound represented by Chemical Formula 1 may be one or more compounds selected from the group consisting of compounds of Chemical Formula D1 to D10, or a pharmaceutically acceptable salt thereof.

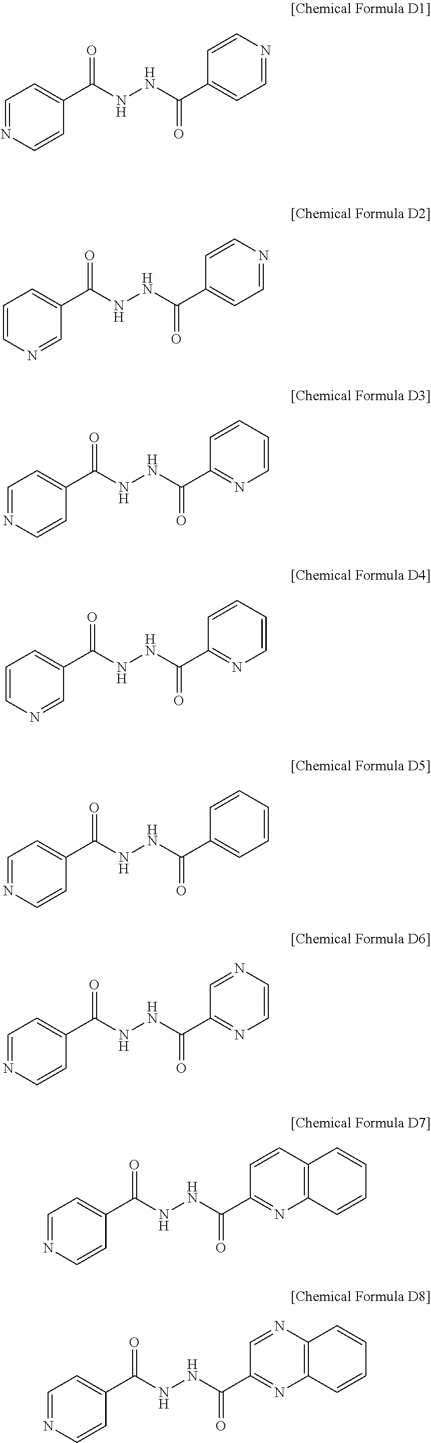

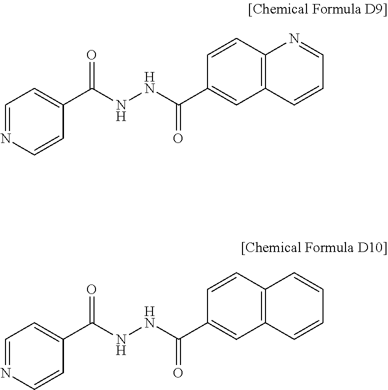 [SMILES: O=C(NNC(=O)c1ccc2ncccc2c1)c1ccncc1 ; O=C(NNC(=O)c1ccc2ccccc2c1)c1ccncc1]

In the present disclosure, chemical formulas D1 to D10 are each used interchangeably with INH-D1 to INH-D10.

Furthermore, according to another aspect of the present disclosure, there is provided a pharmaceutical composition for treating tuberculosis infection, comprising, as an active ingredient, a compound represented by Chemical Formula 1 or a pharmaceutically acceptable salt thereof.

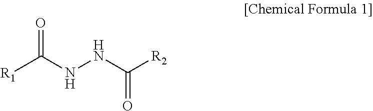

In Chemical Formula 1, R₁ and R₂ are independently selected from the group consisting of nitrogen-containing C₃-C₉ heteroaryl groups and C₆-C₁₂ aryl groups.

All descriptions relating to the compound apply equally to the compound of Chemical Formula 1.

More specifically, the compound represented by Chemical Formula 1 may be at least one compound selected from the group consisting of compounds represented by Chemical Formula D1 to D10 described above, or a pharmaceutically acceptable salt thereof.

In this case, the pharmaceutical composition may include the compound of Chemical Formula 1 or a salt thereof of the present disclosure in an amount of from 0.00001 wt % to 90 wt %, based on the total weight of the pharmaceutical composition, for example, from 0.001 wt % to 50 wt %, preferably from 0.01 wt % to 10 wt %, but the effective amount is not particularly limited to these ranges.

In the present disclosure, the tuberculosis may be derived from Mycobacterium strains, for example, the Mycobacterium strain may be one or more strains selected from the group consisting of Mycobacterium tuberculosis erdman, Mycobacterium tuberculosis, Mycobacterium bovis, Mycobacterium africanum, Mycobacterium microti, and Mycobacterium fortuitum,

Mycobacterium avium, Mycobacterium xenopi, Mycobacterium smegmatis, Mycobacterium tuberculosis H37Rv, Mycobacterium tuberculosis K, Mycobacterium kansasii, Mycobacterium marinum, and Mycobacterium chelonae.

Meanwhile, in the present disclosure, the tuberculosis may be extrapulmonary tuberculosis occurring in at least one of the lymph nodes, the gastrointestinal tract, the joints, the meninges, and the genitourinary tract, and/or pulmonary tuberculosis, for example, it may be one or more selected from the group consisting of ocular tuberculosis, cutaneous tuberculosis, renal tuberculosis, tuberculous lymphadenitis, laryngeal tuberculosis, intestinal tuberculosis, pulmonary tuberculosis, biliary tuberculosis, skeletal tuberculosis, pharyngeal tuberculosis, breast tuberculosis, and spinal tuberculosis. In particular, the tuberculous infection may be at least one selected from the group consisting of ocular tuberculosis, cutaneous tuberculosis, renal tuberculosis, tuberculous lymphadenitis, laryngeal tuberculosis, intestinal tuberculosis, pulmonary tuberculosis, biliary tuberculosis, skeletal tuberculosis, pharyngeal tuberculosis, breast tuberculosis, and spinal tuberculosis.

More particularly, in the present disclosure, the pharmaceutical composition for treating tuberculosis infection may be for treating not only tuberculosis but also related tuberculosis-associated conditions such as tuberculosis-associated inflammation, granulomas, and the like, wherein the tuberculosis-associated inflammation may be at least one selected from the group consisting of chronic inflammation and granuloma.

More specifically, the isoniazid derivative compound represented by Chemical Formula 1, or a salt thereof, included as an active ingredient in the pharmaceutical composition of the present disclosure, may inhibit inflammatory mediators induced by TDM (Trehalose-6,6′-dimycolate). The inflammatory mediator may be at least one of TNF-α and IL-6.

The pharmaceutical composition may be formulated by conventional methods into various forms, including oral formulations such as powders, granules, tablets, capsules, suspensions, emulsions, syrups, and aerosols, as well as sterile injection solutions, and may be administered orally or by various routes including intravenous, intraperitoneal, subcutaneous, intramuscular, intrathecal, rectal, or topical administration and preferably, it is formulated as an injectable preparation to be administered via a route selected from the group consisting of intravenous, intraperitoneal, subcutaneous, intramuscular, and intrathecal.

Examples of suitable carriers, excipients and diluents that may be included in the pharmaceutical composition include lactose, dextrose, sucrose, sorbitol, mannitol, xylitol, erythritol, maltitol, starch, acacia gum, alginate, gelatin, calcium phosphate, calcium silicate, cellulose, methyl cellulose, amorphous cellulose, polyvinyl pyrrolidone, water, methylhydroxybenzoate, propylhydroxybenzoate, talc, magnesium stearate and mineral oil.

In addition, the pharmaceutical composition may further include a filler, an anti-caking agent, a lubricant, a wetting agent, a flavoring agent, an emulsifying agent, a preservative, or the like.

Solid preparations for oral administration include tablets, pills, powders, granules, capsules, and the like, and such solid preparations may be formulated by mixing at least one excipient, for example, starch, calcium carbonate, sucrose, lactose, gelatin, and the like, into the pharmaceutical composition for therapeutic use. In addition to simple excipients, lubricants such as magnesium stearate and talc may also be used. Liquid preparations for oral administration include suspensions, solutions, emulsions, syrups, and the like, and, in addition to commonly used simple diluents such as water and liquid paraffin, may include various excipients such as wetting agents, sweetening agents, flavoring agents, preservatives, and the like.

Formulations for non-oral administration include sterile aqueous solutions, non-aqueous solvents, suspensions, emulsions, lyophilized preparations, and suppositories. Non-aqueous solvents, suspension may include propylene glycol, polyethylene glycol, vegetable oils such as olive oil, and injectable esters such as ethyl oleate. Vehicles for injectable formulations may include conventional additives such as solvents, solubilizers, suspending agents, emulsifying agents, stabilizers, and preservatives.

The appropriate dosage of the pharmaceutical composition of the present disclosure may vary depending on the patient's condition, weight, age, severity of the disease, dosage form, route of administration, and duration of administration, and can be appropriately selected by those skilled in the art.

However, the dosage may vary depending on the route of administration, the severity of the disease, sex, weight, age, or the like, and thus the above dosage does not limit the scope of the present disclosure.

The tuberculosis for which the pharmaceutical composition of the present disclosure for treating tuberculosis infection that can exert a therapeutic effect may be isoniazid-resistant tuberculosis (INHr-TB).

The pharmaceutical composition of the present disclosure may be administered together with a known therapeutic agent for tuberculosis, that is, the pharmaceutical composition for treating a tuberculosis infection may be used in combination with an anti-tuberculosis agent.

More specifically, the pharmaceutical composition of the present disclosure may be administered as a single therapeutic agent or in combination with other therapeutic agents or therapeutic methods, and may be administered concurrently or sequentially with conventional therapeutic agents, for example, the pharmaceutical composition for treating tuberculosis infection may be administered before, after, or concomitantly with an anti-tuberculosis agent. In addition, the pharmaceutical composition of the present disclosure may be administered as a single dose or in multiple doses. Considering all of the above factors, it is important to administer an amount that achieves the maximum effect with a minimal dose and without side effects, which can be readily determined by those skilled in the art.

In this case, as a conventional therapeutic agent, the anti-tuberculosis agent may be one or more selected from the group consisting of pyrazinamide, isoniazid, streptomycin, para-aminosalicylic acid, neomycin, viomycin, kanamycin, and rifampin.

According to another aspect of the present disclosure, there is provided a method of treating tuberculosis comprising the steps of administering the pharmaceutical composition of the present disclosure to a patient with tuberculosis.

The pharmaceutical composition of the present disclosure may be administered to a patient with tuberculosis in a pharmaceutically effective amount.

In the present disclosure, “administration” means providing a given substance to a patient by any suitable means, and the route of administration of the composition of the present disclosure may be any general route capable of delivering the composition to a target tissue, including oral or non-oral routes (e.g., parenteral), preferably oral.

As used herein, the term “patient” refers to a human and to any other animal having a disease whose symptoms may be improved by administering the composition of the present disclosure. The composition according to the present disclosure can be applied not only to humans (for treatment, inhibition, or prevention) but also to other commercially useful animals. For example, it may be animals excluding humans.

As used herein, the term “pharmaceutically effective amount” refers to an amount sufficient to treat a disease at a reasonable benefit/risk ratio applicable to medical treatment, and the level of such an effective amount may be determined based on factors well known in the medical arts, including the type of the patient's disease, severity, the drug's activity, the patient's sensitivity to the drug, the timing of administration, the route and excretion rate of administration, the treatment duration, co-administered drugs, and other clinically recognized factors.

The composition for treating tuberculosis infection of the present disclosure not only effectively suppresses general inflammatory responses but can also be effectively applied as a host-directed therapy to alleviate pulmonary granulomas during tuberculosis treatment.

Embodiments of the present disclosure will now be described in detail. The following embodiments are provided merely for illustrative purposes to facilitate understanding of the present disclosure and are not intended to limit the scope of the disclosure.

### EMBODIMENTS

### 1. Irradiation of Isoniazid and LC-MS Analysis.

After dissolving isoniazid at 5 mg/mL in distilled water, the solution was irradiated in a cobalt-60 gamma irradiation facility (IR-221) at a dose rate of 10 kGy per hour to a total absorbed dose of 50 kGy. Then, structural changes were confirmed and analyzed using ultra-performance liquid chromatography-mass spectrometry (Thermo Ultimate 3000 UPLC system, Thermo LTQ-Orbitrap XL). The column was an ACQUITY BEH C18, 1.7 μm, 150×2.1 mm, solvent A was distilled water containing 0.1% formic acid, and solvent B was methanol. The gradient conditions for analysis condition were set to 0 min (5%), 1.5 min (5%), 10 min (80%), 10.1 min (100%), 12 min (100%), 12.1 min (5%), and 15 min (5%) based on solvent B. Mass spectrometry conditions were ESI in positive mode with a spray voltage of 4.0 kV and a capillary temperature of 300° C.

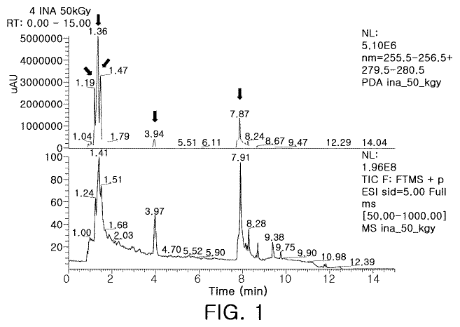

LC-MS analysis of isoniazid irradiated with 50 kGy of gamma rays showed five peaks that were generated. As shown in FIG. 1, the molecular weight of the first peak was 124.04, the second peak was 138.06, the third peak was 123.05, the fourth peak was 243.09, and the fifth peak was 227.09.

The molecular weight of each of these compounds was confirmed to match that of isonicotinic acid, isoniazid, isonicotinamide, and N′-(pyridyl-4-carbonyl)-hydrazide, which have been reported as compounds of a photo-Fenton reaction products of isoniazid reported in a previous paper, and on this basis it was concluded that their structures were identical. Among these, the fourth peak was identified as N′-(pyridyl-4-carbonyl)-hydrazide (hereinafter referred to as “INH-D1”), and, as shown in FIG. 1, isonicotinic acid, isonicotinamide, and one unidentified compound were found to be present in addition to isoniazid.

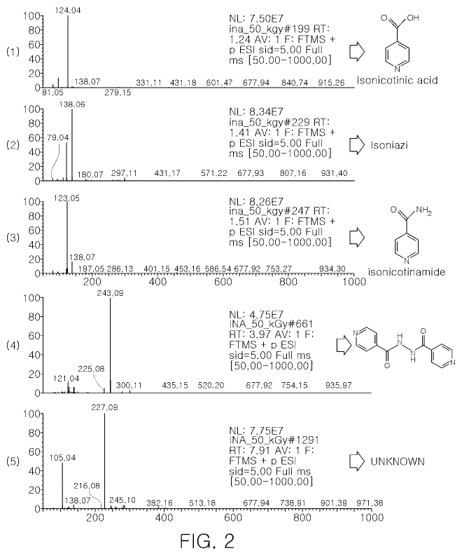

FIG. 2 illustrates compounds corresponding to each peak identified above.

### 2. Synthesis of Isoniazid Derivatives.

Additional isoniazid derivatives having structures similar to INH-D1, which was identified following irradiation of isoniazid in embodiment 1, were synthesized. Isonicotinic acid and isonicotinamide have been previously reported as antibiotics, but are known to be ineffective for treating tuberculosis.

### (1) Synthesis of INH-D1

Isoniazid (137 mg, 1 mmol) was dissolved in pyrimidine (8 mL), and isonicotinoyl chloride hydrochloride (141 mg, 1 mmol) was added to the mixture at 0° C. and after the addition of all reagents, the mixture was stirred at 60° C. for 12 hours. When the reaction was complete, the mixture was cooled to room temperature and washed five times with distilled water at 0° C. After washing was completed, the solid remaining on the filter was collected, and the solvent was removed under reduced pressure and INH-D1 (80 mg, 33%) was obtained.

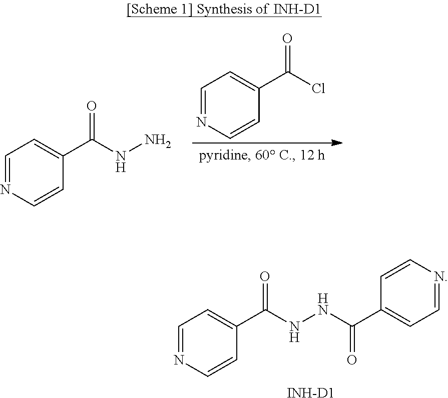

### (2) INH-D2 Synthesis

Nicotinic hydrazide (480 mg, 3.5 mmol) and K₂CO₃ (690 mg, 5 mmol) were sufficiently dissolved in DMF (10 mL), and the solution was cooled to 0° C. Then, isonicotinoyl chloride hydrochloride (890 mg, 5 mmol) was added, and the mixture was stirred at 70° C. for 12 hours. When the reaction was complete, the mixture was cooled to room temperature and washed five times with distilled water at 0° C. After washing was completed, the solid remaining on the filter was collected, and the solvent was removed under reduced pressure and INH-D2 (611 mg, 72%) was obtained.

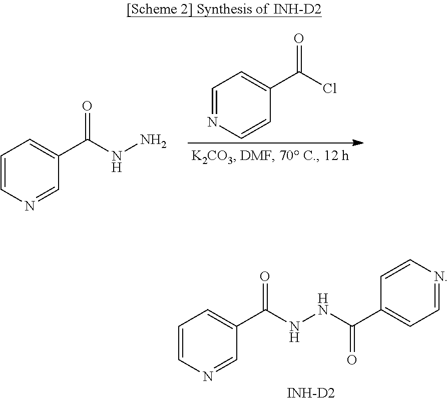

### (3) Synthesis of INH-D3

Isoniazid (342 mg, 2.5 mmol) and K₂CO₃ (553 mg, 4 mmol) were sufficiently dissolved in DMF (10 mL), and the solution was cooled to 0° C. Then, 2-pyridine carbonyl chloride hydrochloride (553 mg, 3 mmol) was added, and the mixture was stirred at 50° C. for 12 hours. When the reaction was complete, the mixture was cooled to room temperature and washed five times with distilled water at 0° C. After the washing, the solid remaining on the filter was collected, the solvent was removed under reduced pressure, and INH-3 (251 mg, 41%) was obtained.

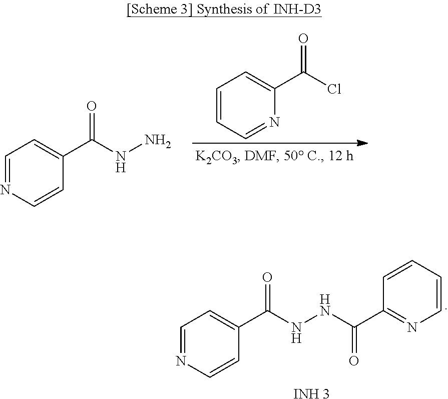 [SMILES: NNC(=O)c1ccncc1 ; O=C(Cl)c1ccccn1 ; O=C(NNC(=O)c1ccccn1)c1ccncc1 ; "INH 3"]

### (4) Synthesis of INH-D4

Nicotinic hydrazide (480 mg, 3.5 mmol) and K₂CO₃ (690 mg, 5 mmol) were sufficiently dissolved in DMF (10 mL), and the solution was cooled to 0° C. Then, 2-pyridine carbonyl-chloride hydrochloride (890 mg, 5 mmol) was added, and the mixture was stirred at room temperature for 12 hours. When the reaction was complete, the mixture was washed five times with distilled water at 0° C. After the washing was completed, the solid remaining on the filter was collected, and the solvent was removed under reduced pressure and INH-4 (500 mg, 59%) was obtained.

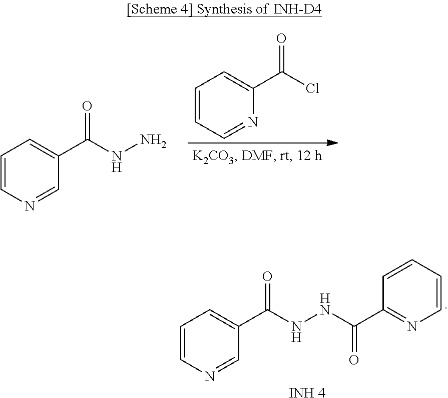 [SMILES: NNC(=O)c1cccnc1 ; O=C(Cl)c1ccccn1 ; O=C(NNC(=O)c1ccccn1)c1cccnc1 ; "INH 4"]

### (5) INH-D5 Synthesis

Isoniazid (823 mg, 6 mmol) was dissolved in tetrahydrofuran (5 mL), then triethylamine (1 mL, 12.3 mmol) was added, and the solution was cooled to 0° C. Then, benzoyl chloride (348 μL, 3 mmol) dissolved in 1 mL of tetrahydrofuran was added, and the mixture was stirred at 70° C. for 12 hours. When the reaction was complete, the mixture was washed five times with distilled water at 0° C. After washing, the solid remaining on the filter was collected, and the solvent was removed under reduced pressure and INH-5 (360 mg, 25%) was obtained.

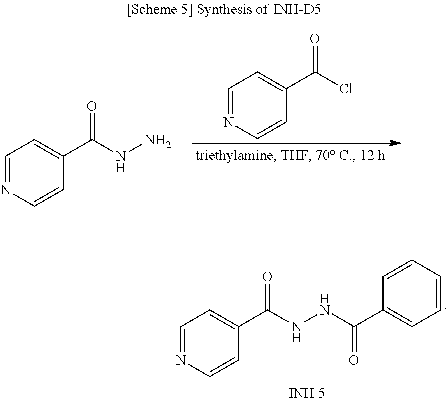 [SMILES: NNC(=O)c1ccncc1 ; O=C(Cl)c1ccncc1 ; O=C(NNC(=O)c1ccncc1)c1ccccc1 ; "INH 5"]

### (6) Synthesis of INH-D6

Pyrazinoic acid (148.92 mg, 1.20 mmol) was dissolved in a mixed solvent of tetrahydrofuran (THF, 7 mL) and dichloromethane (DCM, 7 mL), and 1-(3-dimethylaminopropyl)-3-ethylcarbodiimide hydrochloride (EDC·HCl, 230.04 mg, 1.20 mmol) was added. Then isoniazid (137.14 mg, 1.00 mmol) and 4-dimethylaminopyridine (DMAP, 36.65 mg, 0.30 mmol), each dissolved in THF (1 mL) and DCM (1 mL), were added to the reaction mixture, and the mixture was stirred at room temperature for 72 hours. When the reaction was complete, the mixture was purified by MPLC (DCM/MeOH) and INH-6 (88.3 mg, 36%) was obtained.

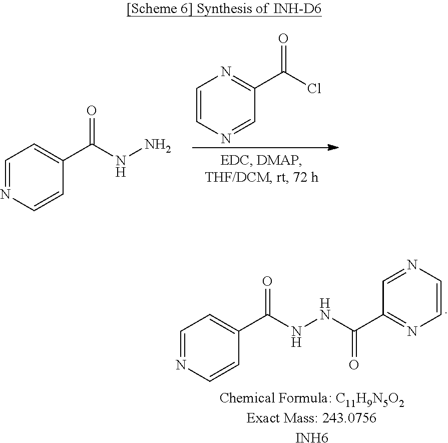 [SMILES: NNC(=O)c1ccncc1 ; O=C(Cl)c1cnccn1 ; O=C(NNC(=O)c1cnccn1)c1ccncc1 ; "Chemical Formula: C11H9N5O2Exact Mass: 243.0756INH6"]

### (7) Synthesis of INH-D7

Quinaldic acid (207.80 mg, 1.20 mmol) was dissolved in a mixed solvent of tetrahydrofuran (THF, 5 mL) and dichloromethane (DCM, 5 mL), and then 1-(3-dimethylaminopropyl)-3-ethylcarbodiimide hydrochloride (EDC·HCl, 230.04 mg, 1.20 mmol) was added. Subsequently, isoniazid (137.14 mg, 1.00 mmol) and 4-dimethylaminopyridine (DMAP, 36.65 mg, 0.30 mmol) dissolved in THF (1 mL) and DCM (1 mL) were added to the reaction mixture, and the mixture was stirred at room temperature for 72 hours. After completion of the reaction, the mixture was purified by MPLC (DCM/MeOH) and INH-7 (227 mg, 78%) was obtained.

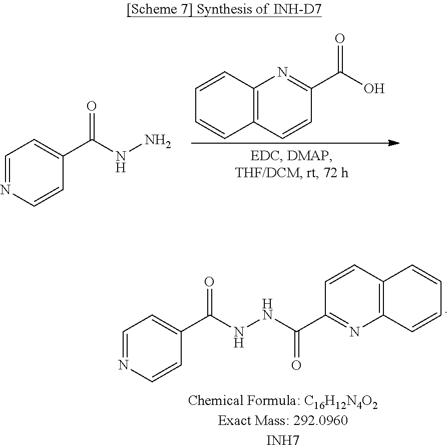 [SMILES: NNC(=O)c1ccncc1 ; O=C(O)c1ccc2ccccc2n1 ; O=C(NNC(=O)c1ccc2ccccc2n1)c1ccncc1 ; "Chemical Formula: C16H12N4O2Exact Mass: 292.0960INH7"]

### (8) Synthesis of INH-D8

2-Quinoxaline carboxylic acid (174.04 mg, 1.00 mmol) was dissolved in a mixed solvent of tetrahydrofuran (THF, 8 mL) and dichloromethane (DCM, 8 mL), and then 1-(3-dimethylaminopropyl)-3-ethylcarbodiimide hydrochloride (EDC·HCl, 230.04 mg, 1.20 mmol) was added. Subsequently, isoniazid (164.57 mg, 1.20 mmol) and 4-dimethylaminopyridine (DMAP, 36.65 mg, 0.30 mmol) dissolved in THF (2 mL) and DCM (2 mL) were added to the reaction mixture, and the mixture was stirred at room temperature for 72 hours. After the reaction was complete, the mixture was purified by MPLC (DCM/MeOH) to obtain INH-8 (190 mg, 65%).

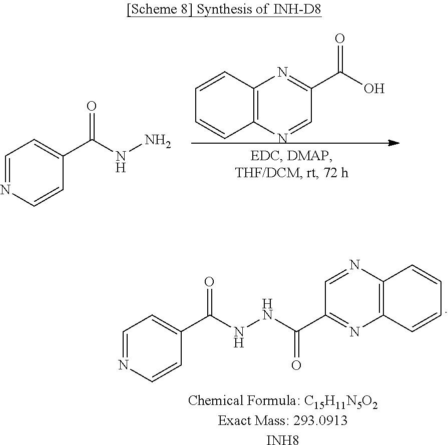 [SMILES: NNC(=O)c1ccncc1 ; O=C(O)c1cnc2ccccc2n1 ; O=C(NNC(=O)c1cnc2ccccc2n1)c1ccncc1 ; "Chemical Formula: C15H11N5O2Exact Mass: 293.0913INH8"]

### (9) INH-D9 Synthesis

6-Quinoline carboxylic acid (519.14 mg, 3.00 mmol) was dissolved in a mixed solvent of tetrahydrofuran (THF, 8 mL) and dichloromethane (DCM, 8 mL), after that 1-ethyl-3-(3-dimethylaminopropyl) carbodiimide hydrochloride (EDC·HCl, 766.8 mg, 4.00 mmol) was added. Then isoniazid (548.56 mg, 4.00 mmol) and 4-dimethylaminopyridine (DMAP, 122.17 mg, 1.00 mmol) dissolved in THF (2 mL) and DCM (2 mL) were added to the reaction mixture, and the mixture was stirred at room temperature for 72 hours. After completion of the reaction, the mixture was purified by using MPLC (DCM/MeOH) to obtain INH-9 (55 mg, 6%).

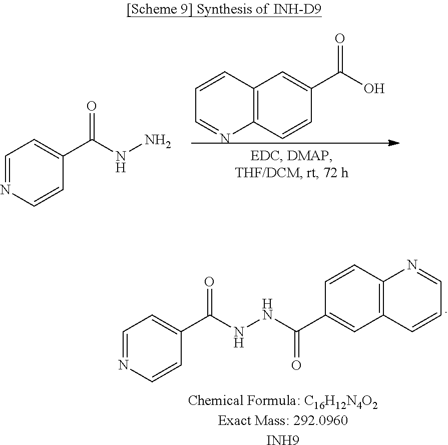 [SMILES: NNC(=O)c1ccncc1 ; O=C(O)c1ccc2ncccc2c1 ; O=C(NNC(=O)c1ccc2ncccc2c1)c1ccncc1 ; "Chemical Formula: C16H12N4O2Exact Mass: 292.0960INH9"]

### (10) Synthesis of INH-D10

2-Naphthoic acid (516.54 mg, 3.00 mmol) was dissolved in a mixed solvent of tetrahydrofuran (THF, 8 mL) and dichloromethane (DCM, 8 mL), and 1-(3-dimethylaminopropyl)-3-ethylcarbodiimide hydrochloride (EDC·HCl, 766.8 mg, 4.00 mmol) was added. Subsequently, isoniazid (548.56 mg, 4.00 mmol) and 4-dimethylaminopyridine (DMAP, 122.17 mg, 1.00 mmol) dissolved in THF (2 mL) and DCM (2 mL) were added to the reaction mixture, and the mixture was stirred at room temperature for 72 hours. After completion of the reaction, the mixture was purified by MPLC (DCM/MeOH) to obtain INH-10 (392.3 mg, 45%).

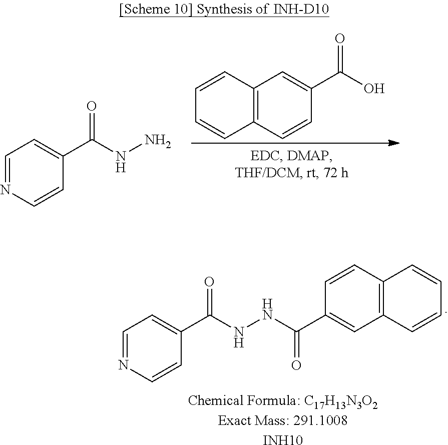 [SMILES: NNC(=O)c1ccncc1 ; O=C(O)c1ccc2ccccc2c1 ; O=C(NNC(=O)c1ccc2ccccc2c1)c1ccncc1 ; "Chemical Formula: C17H13N3O2Exact Mass: 291.1008INH10"]

### 3. Structural Confirmation of INH-D1 to INH-D10.

To confirm the structures of the synthesized INH-D1 to INH-D10 compounds, nuclear magnetic resonance (NMR) analysis was performed following synthesis.

As a result, the following ¹H NMR and ¹³C NMR data were observed for each compound.

Analytical data for INH-1: ¹H NMR (500 MHz, DMSO-d6) δ 10.99 (s, 2H), 8.81 (d, J=5.5 Hz, 4H), 7.84 (d, J=5.6 Hz, 4H); ¹³C NMR (125 MHz, DMSO-d6) δ 164.3, 150.5, 139.3, 121.4.

Analytical data for INH-2: ¹H NMR (500 MHz, DMSO-d6) δ 10.90 (s, 2H), 8.81 (m, 3H), 8.27 (d, J=7.62 Hz, 1H), 7.84 (d, J=4.61 Hz, 2H), 7.58 (dd, J=4.98, 2.15 Hz, 1H); ¹³C NMR (125 MHz, DMSO-d6) δ 164.9, 164.8, 153.1, 151.0, 148.9, 139.9, 165.7, 128.5, 124.2, 121.8.

Analytical data for INH-3: ¹H NMR (500 MHz, DMSO-d6) δ 10.86 (s, 1H), 10.77 (s, 1H), 8.79 (dd, J=6.05, 1.65 Hz, 2H), 8.72-8.71 (m, 1H), 8.08-8.03 (m, 2H), 7.82 (dd, J=6.05, 1.6 Hz, 2H), 7.69-7.66 (m, 1H); ¹³C NMR (125 MHz, DMSO-d6) δ 163.9, 163.2, 150.4, 149.1, 148.7, 139.5, 137.9, 127.1, 122.4, 121.3.

Analytical data for INH-4: ¹H NMR (500 MHz, DMSO-d6) δ 10.77 (br s, 1H), 10.73 (br s, 1H), 9.07 (d, J=1.6, 1H), 8.78 (m, 1H), 8.72 (d, J=4.55, 1H), 8.27-8.25 (m, 1H), 8.08-8.03 (m, 2H), 7.69-7.66 (m, 1H), 7.59-7.56 (m, 1H); ¹³C NMR (500 MHz, DMSO-d6) δ 164.1, 163.3, 152.5, 149.1, 148.7, 148.5, 137.9, 135.3, 128.2, 127.1, 123.7, 122.5.

Analytical data for INH-5: ¹H NMR (500 MHz, DMSO-d6) δ 10.73 (br s, 2H), 8.80 (dd, J=4.25, 1.15 Hz, 2H), 7.93 (m, 2H), 7.83 (dd, J=4.3, 1.15 Hz, 2H), 7.61 (m, 1H), 7.53 (m, 2H); ¹³C NMR (500 MHz, DMSO-d6) δ 165.8, 164.4, 150.6, 150.5, 137.5, 132.4, 132.0, 128.6, 128.5, 127.5, 121.4.

Analytical data for INH-6: ¹H NMR (500 MHz, DMSO-d6) δ 10.95 (d, J=24.45, 2H), 9.22 (d, J=1.45, 1H), 8.95 (d, J=2.5, 1H), 8.81-8.82 (m, 1H), 8.80 (m, 2H), 7.82-7.83 (m, 2H); ¹³C NMR (500 MHz, DMSO-d6) δ 163.99, 162.30, 150.50, 148.14, 144.07, 143.77, 143.69, 139.41, 121.34.

Analytical data for INH-7: ¹H NMR (500 MHz, DMSO-d6) δ 10.96 (s, 2H), 8.81-8.82 (m, 2H), 8.62 (d, J=8.3 Hz, 1H), 8.16 (t, J=15.5 Hz, 1H), 8.11 (d, J=0.8 Hz, 1H), 7.90-7.93 (m, 1H), 7.85-7.86 (m, 2H), 7.75-7.78 (m, 1H); ¹³C NMR (500 MHz, DMSO-d6) δ 164.11, 163.44, 150.51, 149.17, 146.09, 139.54, 138.04, 130.75, 129.31, 129.00, 128.44, 128.20, 121.39, 118.87.

Analytical data for INH-8: ¹H NMR (500 MHz, DMSO-d₆) δ 11.10 (d, J=58.9, 2H), 9.49 (s, 1H), 8.81-8.82 (m, 2H), 8.22-8.26 (m, 2H), 8.00-8.06 (m, 2H), 7.85-7.86 (m, 2H); ¹³C NMR (500 MHz, DMSO-d₆) δ 164.14, 162.57, 150.53, 143.56, 143.52, 143.16, 139.84, 139.42, 132.35, 131.56, 129.58, 129.20, 121.39.

Analytical data for INH-9: ¹H NMR (500 MHz, DMSO-d6) δ 10.95 (s, 1H), 9.02 (s, 1H), 8.82 (d, J=3.6, 2H), 8.65 (d, J=14.15, 1H), 8.53 (d, J=7.45, 1H), 8.25 (d, J=8.5, 1H), 8.16 (d, J=8.45, 1H), 7.87 (d, J=3.65, 2H), 7.63 (d, J=3.6, 1H); ¹³C NMR (500 MHz, DMSO-d₆) δ 165.46, 164.42, 152.48, 150.55, 148.92, 139.52, 137.30, 130.21, 129.41, 128.68, 127.61, 127.17, 122.41, 121.40.

Analytical data for INH-10: ¹H NMR (500 MHz, DMSO-d₆): δ 10.84 (s, 2H), 8.82-8.80 (m, 2H), 8.56 (s, 1H), 8.09-8.06 (m, 2H), 8.02 (d, J=8, 1H), 8.00-7.98 (m, 1H), 7.85 (m, 2H), 7.65 (m, 2H); ¹³C NMR (500 MHz, DMSO-d₆): δ 165.84, 164.46, 150.52, 139.55, 134.45, 132.08, 129.69, 129.00, 128.23, 128.12, 128.02, 127.71, 126.97, 123.99, 121.35.

As a result, the synthesized compounds INH-D1 to INH-D10 were confirmed to be the following compounds.
INH-D1: N′-isonicotinoyl-isonicotinohydrazide
INH-D2: N′-isonicotinoyl-nicotinohydrazide
INH-D3: N′-isonicotinoyl-picolinohydrazide
INH-D4: N′-nicotinoyl-picolinohydrazide
INH-D5: N′-benzoy-lisonicotinohydrazide
INH-D6: N′-isonicotinoylpyrazine-2-carbohydrazide.
INH-D7: N′-isonicotinoylquinoxaline-2-carbohydrazide
INH-D8: N′-isonicotinoylquinoline-3-carbohydrazide.
INH-D9: N′-isonicotinoylquinoline-6-carbohydrazide.
INH-D10: N′-isonicotinoyl-2-naphthohydrazide.

### 4. Cytotoxicity Evaluation of Isoniazid Derivatives

The mouse macrophage cell line (RAW264.7) was cultured in high-glucose DMEM (Dulbecco's modified Eagle's medium; WelGene) supplemented with 10% FBS (fetal bovine serum; Gibco BRL) and 100 U/mL penicillin/streptomycin (Invitrogen) at 37° C. in an incubator containing 5% Co₂.

To evaluate cytotoxicity, isoniazid (INH) and its derivatives (INH-D1 to INH-D5) were applied at varying concentrations, and after 24 hours, the cells were stained with 0.5 mg/mL MTT (3-(4,5-Dimethylthiazol-2-yl)-2,5-Diphenyltetrazolium Bromide) reagent to assess cell viability.

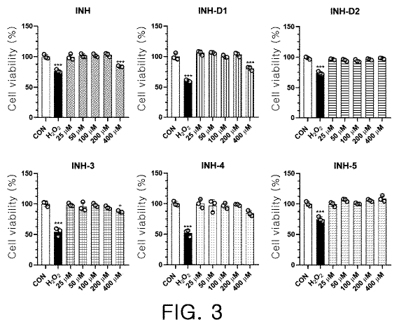

As a result of the cell viability experiment, INH, INH-D1, and INH-3 exhibited no cytotoxicity at concentrations of 200 μM or less, and INH-D2, INH-4, and INH-D5 exhibited no cytotoxicity at concentrations up to 400 μM (FIG. 3).

Based on this, it was confirmed that when each derivative was administered together with TDM (trehalose 6,6′-dimycolate) derived from tuberculosis, neither INH nor the INH derivatives reduced cell viability at concentrations of 200 μM or less.

5. In Vitro Evaluation of the Inhibitory Efficacy of Isoniazid Derivatives on Inflammatory Factors Associated with Pulmonary Granulomas.

The inhibitory efficacy against inflammatory mediators associated with pulmonary granuloma formation was evaluated by measuring cytokine levels using ELISA assay and by analyzing surface molecule expression using flow cytometer.

### (1) Measurement of Cytokines According to the Concentrations of INH and INH-D1 to INH-D5 Derivatives.

50 μg of TDM (Trehalose-6,6′-dimycolate) derived from tuberculosis were dissolved in 1 mL of a hexane-ethanol mixture (v/v 9:1), dispensed into 48-well plates at 1 μg per well, and coated by evaporating the organic solvent in a laminar flow hood. RAW264.7 cells were seeded at a density of 1×10⁵ cells/well in TDM-coated 48-well plates and cultured for 2 hours.

Then, INH, INH-D1, INH-D2, INH-3, INH-4, and INH-5 were each treated at concentrations of 50, 100, and 200 μM. The cells were further incubated for 18 hours at 37° C. in a 5% CO₂ environment. After incubation, the supernatants were collected, and the concentrations of the cytokines TNF-α (Cat. 555268), IL-6 (Cat. 555540), and IL- 12p70 (Cat. 555256) were measured using ELISA assay kits. All subsequent procedures were performed according to the manufacturer's instructions of BD Biosciences.

TNF-α plays an important role in the formation and maintenance of pulmonary granulomas and contributes to recruiting and activating immune cells to contain and eliminate Mycobacterium tuberculosis. IL-6 amplifies inflammatory responses and activates immune cells, thereby enhancing the immune response against tuberculosis. However, the amplification of these inflammatory responses leads to tissue damage and functional impairment, and in particular, the formation of pulmonary granulomas can drive Mycobacterium tuberculosis into a dormant state, which can lead to resistance and recurrent tuberculosis.

It was confirmed that the production of inflammatory cytokines decreased in a concentration-dependent manner at all concentrations (50, 100, and 200 μM) of the compounds of the present disclosure.

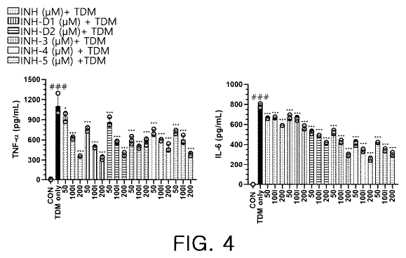

Compared to INH at the same concentration, INH-D1 to INH-D5 exhibited superior IL-6 and TNF-α inhibitory efficacy (FIG. 4).

### (2) Measurement of Cytokines According to the Concentrations of INH and INH-D1 to INH-D10 Derivatives.

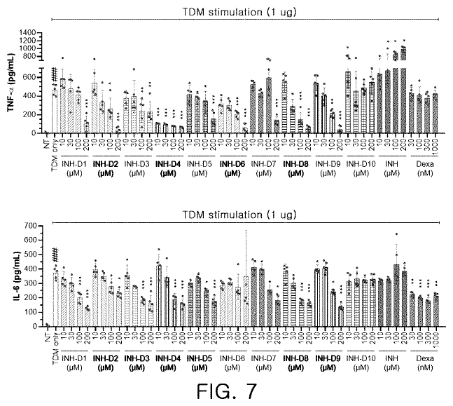

50 μg of TDM (Trehalose-6,6′-dimycolate) derived from tuberculosis were dissolved in 1 mL of a hexane and ethanol mixture (9:1 by volume), dispensed into a 48-well plate at 1 μg per well, and coated by evaporating the organic solvent in a laminar flow hood. RAW264.7 cells were seeded at a density of 1×10⁵ cells/well in TDM-coated 48-well plates and cultured for 2 hours. Thereafter, INH derivatives (INH-D1 to INH-D10), INH, and Dexa were applied at various concentrations, and the cells were incubated for 18 hours at 37° C. in an atmosphere of 5% CO₂. After 18 hours of incubation, cell supernatants were collected, cytokine levels in the supernatants were measured using ELISA assay kits, and the results are shown in FIG. 7.

### (3) Analysis of Surface Molecules

RAW264.7 cells were seeded at a density of 1×10\hat{ }5 cells/well in TDM-coated 48-well plates and cultured for 2 hours. Then, INH, INH-D1, INH-D2, INH-3, INH-4, and INH-5 were each treated at concentrations of 50, 100, and 200 μM. The cells were further cultured for 18 hours at 37° C. in a 5% CO₂ atmosphere. After incubation, the cells were harvested (125×g, 5 minutes) and washed with PBS. The cells were then incubated in the dark for 15 minutes with antibodies against surface molecules. The antibodies used were FITC-labeled anti-CD80 (Cat. 561954, BD Biosciences), PE-labeled anti-CD86 (Cat. 553692, BD Biosciences), APC-labeled anti-MHC-I (Cat. 17-5958-82, Invitrogen, Waltham, MA, USA), PE-Cyanine7 (Cy7)-labeled anti-MHC-II (Cat. 25-5321-82, Invitrogen), and BV510-Live/Dead (Cat. L34966, Invitrogen). After washing with PBS, the cells were analyzed. Cell analysis was performed using a MACSQuant VYB flow cytometer.

CD80 and CD86 are costimulatory molecules expressed on the surface of antigen-presenting cells (APC), inducing full activation of T cells and thereby regulating and amplifying inflammatory responses. MHC class I presents intracellular antigens to CD8+ T cells (cytotoxic T cells) to enable recognition and elimination of infected or damaged cells, which enhances the cytotoxic response at sites of inflammation and contributes to the rapid clearance of infectious agents. MHC class II is primarily expressed on antigen-presenting cells and presents extracellular antigens to CD4+ T cells (helper T cells) to regulate and amplify the inflammatory response.

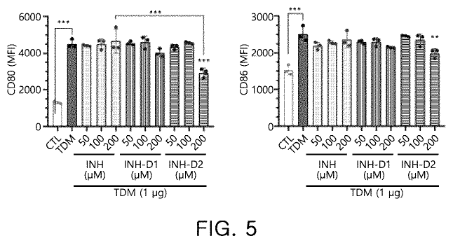

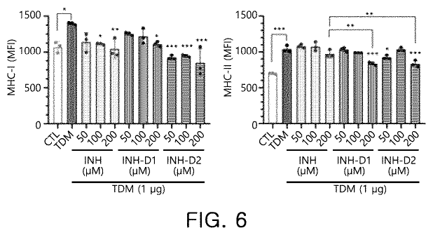

The effect of isoniazid derivatives on the expression of costimulatory molecules and MHC in response to TDM stimulation was evaluated. CD80 and CD86 were significantly inhibited in the INH-D2 200 μM treatment group (FIG. 5). For MHC-I, expression was inhibited by INH at 100 μM and 200 μM, by INH-D1 at 200 μM, and significantly by INH-D2 at all tested concentrations. For MHC-II, INH did not reduce expression, whereas INH-D1 reduced expression at 100 μM and INH-D2 reduced expression at 50 μM and 200 μM (FIG. 6).

### 6. Evaluation of Efficacy in Ameliorating Pulmonary Granulomas in a Mouse Model

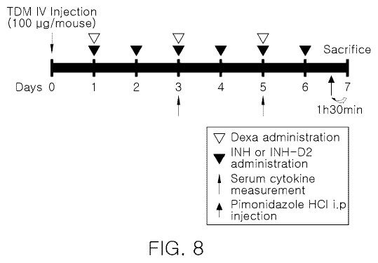

To evaluate whether inhibition of TDM-induced inflammatory mediators by isoniazid derivatives alleviates pulmonary granulomatous lesions, experiments were conducted using a mouse model. The dosing and sacrifice schedule for the animal experiments is shown in FIG. 8.

1 mg of TDM (cord factor, GB61684, GLPBIO) was mixed with 90 μL of mineral oil (cat. 330779, Sigma-Aldrich Co., St. Louis, MO, USA), and then homogenized by mixing with 910 μL of PBS containing Tween-80. In 9-week-old mice, 100 μL of an emulsion containing 100 μg of TDM was injected into the tail vein (100 μg/mouse). The control group was injected with an emulsion solution not containing TDM. INH (25 mg/kg), INH-D1 (5 and 25 mg/kg), and INH-D2 (5 and 25 mg/kg) were administered orally once daily for 5 days starting one day after the TDM intravenous injection. As a positive control, dexamethasone (0.5 mg/kg) was administered orally on day 1, day 3, and day 5. The mice were euthanized seven days after.

### (1) Histopathological Analysis of Lung Tissue

The left lung lobe was fixed in 10% formalin for histopathological examination. Lung tissues were dehydrated, progressively immersed in alcohol and xylene, and embedded in paraffin. Tissue sections (5 μm) that were deparaffinized and stained with hematoxylin and eosin (H&E, #ab245880) at Abcam (Cambridge, UK) were used. Histopathological analysis of lung inflammation was performed using Motic Digital Slide Assistant software version 1.0.7.44 (Kowloon Bay, Kowloon, Hong Kong) (PMID: 31734231). High-resolution images of the H&E-stained slides were scanned. Inflammation was measured in two steps using ImageJ software version 1.52 (NIH, Bethesda, MD; https://imagej.nih.gov/ij). The maximum threshold of all analyzed digital transparency within the lung area was set to 200. The minimum and maximum values of hue, saturation, and brightness were set to 120 and 255; 0, 255; and 0, 255, respectively. Total cell area was determined by modifying the procedure described in the online ImageJ staining examples directory (NIH, https://imagej.nih.gov/ij/docs/examples). Lung inflammation was calculated using the ratio of the total area occupied by cells.

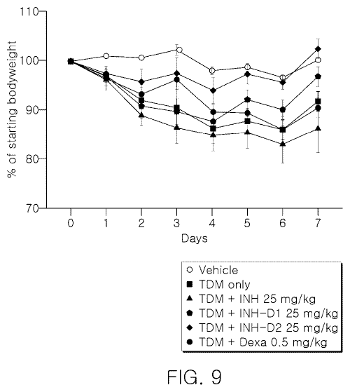

As a result of measuring the body weight of mice for 7 days, the INH-D2 25 mg/kg administration group exhibited significant body weight recovery compared to the lung granuloma induction group (TDM only) (FIG. 9).

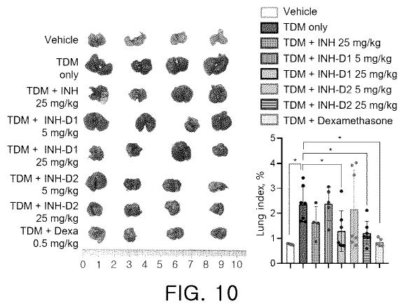

On day 7, the mice were sacrificed, their lungs were excised and weighed, and the lung index (lung weight/body weight×100) was calculated and there was no significant difference between the TDM-only group and the INH 25 mg/kg group. However, the lung index decreased in the INH-D1 25 mg/kg and INH-D2 25 mg/kg groups that received high doses of the derivative. The lung index was also significantly decreased in the dexamethasone-treated group, which served as a positive control. FIG. 10(a) shows photographs of the excised lungs, and FIG. 10(b) presents the lung index as a graph.

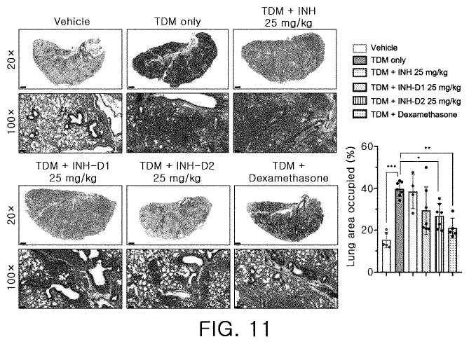

Furthermore, histopathological analysis revealed a clear granulomatous lesion in the TDM-only group compared to the vehicle group, and computer analysis of the histopathological lesions showed a significant increase in the area occupied in the lungs. Alleviation of these lesions was observed in the INH-D2 25 mg/kg administration group and in the dexamethasone administration group, which served as a positive control. FIG. 11(a) shows photographs of the histopathological lesions, and FIG. 11(b) is a graph showing the area occupied by the granulomatous lesions in the lungs.

### (2) Analysis of Pulmonary Immune Cells

Pimonidazole was prepared at 6 mg/mL and administered by intraperitoneal injection 90 minutes before euthanasia of the mice. After the mice were euthanized, the lungs were collected and weighed. Using scissors on a clean bench, the lungs were cut and each lung was immersed in 0.5 mL of digestion buffer. The digestion buffer consisted of RPMI 1640 medium containing 1 mg/mL collagenase IV (Cat. C1639, Sigma-Aldrich Co.) and 0.1 mg/mL DNase I (Cat. 10104159001, Sigma-Aldrich Co.). After incubation at 37° C. for 30 minutes, the suspensions were passed through a 40 μm mesh. The cells were obtained by centrifugation at 1,800 rpm for 3 minutes and then treated with red blood cell lysis buffer (Cat. R7757, Sigma-Aldrich Co.). The cells were neutralized by adding RPMI (containing 10% FBS and 1% penicillin) and passed again through the 40 μm mesh. After centrifugation at 1,800 rpm for 3 minutes, the supernatant was removed and the cells were immersed in PBS. 100 μL of lung single-cell were stained for 15 minutes in the dark with BV421-labeled anti-CD45 (Cat. 563890, BD Biosciences), BV510-labeled anti-Ly6G (Cat. 740157, BD Biosciences), PE-Cy7-labeled anti-MHC-II (Cat. 25-5321-82, eBioscience), PE-labeled anti-CD11b (Cat. 101208, BioLegend), Alexa Fluor 647-labeled anti-CD64 (FcγRI) (Cat. 558539, BD Biosciences), PE-CF594-labeled anti-CD24 (Cat. 562477, BD Biosciences), and an FITC-labeled Hypoxyprobe antibody. The cells were collected by centrifugation at 1,800 rpm for 3 minutes and washed with PBS. The cells were collected again and 400 μL of PBS was added. 30 μL of the cell suspension were analyzed using a MACSQuant VYB flow cytometer.

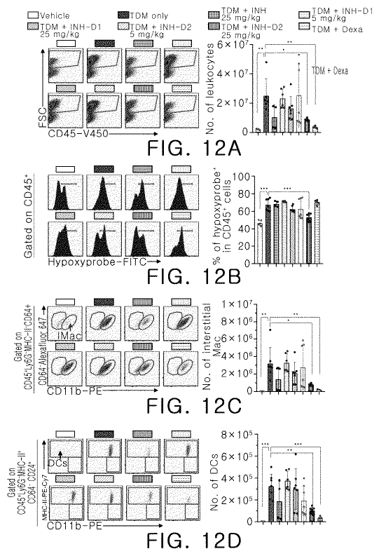

The formation of lung granulomas during tuberculosis infection involves immune cells such as macrophages, lymphocytes, neutrophils, and dendritic cells, and fibroblasts and plasma cells. To determine whether the ameliorative effect of the compound of the present disclosure on granulomatous lesions is substantially associated with cellular infiltration in lung tissue, the cellular composition of lung tissue was analyzed using a flow cytometer. Total leukocytes (CD45+ cells) were decreased in the groups treated with INH-D2 (25 mg/kg) and with dexamethasone (FIG. 12A).

During infiltration of these immune cells, hypoxic conditions develop, causing Mycobacterium tuberculosis to enter a dormant state, which makes drug treatment difficult. Using Hypoxyprobe to assess whether the hypoxic conditions were improved, significant improvement in hypoxia was observed in the INH-D2 25 mg/kg treatment group (FIG. 12B). Subsequently, the numbers of interstitial macrophages and dendritic cells were significantly increased in the INH-D2 25 mg/kg and dexamethasone treatment groups, respectively (FIG. 12C and FIG. 12D).

### (3) Cytokine and Chemokine Analysis in Serum.

Levels of chemokines involved in immune cell migration and of cytokines mediating inflammatory responses were analyzed in serum on days 3 and 5 after induction of pulmonary granulomas.

Retro-orbital blood collected on day 3 and day 5 after TDM administration was centrifuged at 12,000 rpm for 15 minutes to separate serum. To analyze cytokine and chemokine levels in mouse serum, a cytometric bead array kit (Cat. 558266, BD Biosciences) was used to measure IIFN-γ (#558296), IL-2 (#558297), KC (558340), IL-6 (#558301), MCP-1 (558342), IL-10 (#558300), TNF-α (#558299), IL-12p70 (#558303), RANTES (#558345), and IL-1β (#560232)

25 μL of capture beads coated with specific capture antibodies against each cytokine and chemokine (0.5 μL diluted into 25 μL of phosphate buffered saline containing 0.5% BSA, 1 mM EDTA, and 0.05% tween-20) were mixed with 50 μL of serum and incubated for 1 hour in the dark. 25 μL of PE-detection beads (0.5 μL diluted into 25 μL of phosphate-buffered saline containing 0.5% BSA and 5% polyethylene glycol) were added and stained for an additional 1 hour in the dark. After addition of wash buffer, it was centrifuged at 200×g for 5 minutes. After washing, cytokines and chemokines in the serum were analyzed using a MACSQuant VYB flow cytometer (Miltenyi Biotec).

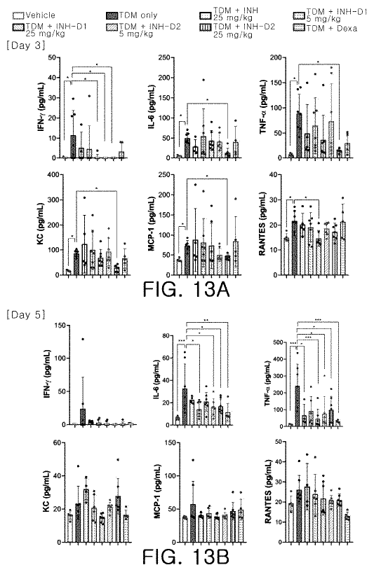

On day 3 after pulmonary granuloma induction, the cytokine IFN-γ was reduced in the INH-D1 25 mg/kg, INH-D2 5 mg/kg, and INH-D2 25 mg/kg groups compared to the TDM-only group. IL-6 and TNF-α were significantly reduced in the INH-D2 25 mg/kg treatment group. The chemokine KC, which induces neutrophil infiltration, and the chemokine MCP-1, which induces macrophage/monocyte infiltration, were also significantly reduced in the INH-D2 25 mg/kg treatment group, whereas no significant differences were observed for RANTES, which induces T-cell and monocyte infiltration, in any of the drug-treated groups (FIG. 13A).

On day 5 after induction of pulmonary granulomas, IL-6 was inhibited in the INH-D1 5 mg/kg, INH-D2 5 mg/kg, INH-D2 25 mg/kg, and dexamethasone-treated groups compared with the TDM-only group. TNF-α was reduced in the INH 25 mg/kg, INH-D1 25 mg/kg, INH-D2 5 mg/kg, INH-D2 25 mg/kg, and dexamethasone-treated groups, and no significant differences were observed for other cytokines and chemokines (FIG. 13B).

### 7. Comparative Study of Isoniazid Dimers INH-D2, D4, D6, and D8.

1 mg of TDM (Cord factor, GB61684, GLPBIO) was mixed with 90 μL of mineral oil (cat. 330779, Sigma-Aldrich Co., St. Louis, MO, USA), and the mixture was homogenized with 910 μL of PBS containing Tween-80. In 9-week-old mice, 100 μL of an emulsion containing 100 μg of TDM was injected into the tail vein (100 μg/mouse). The control group was injected with an emulsion that did not contain TDM. INH-D2, INH-D4, INH-D6, and INH-D8 were each administered orally at a dose of 30 mg/kg once daily for five days, starting one day after the TDM intravenous injection. As a positive control, dexamethasone 0.5 mg/kg was administered intraperitoneally on day 1, day 3, and day 5. The mice were euthanized seven days later.

### (1) Lung Index Analysis

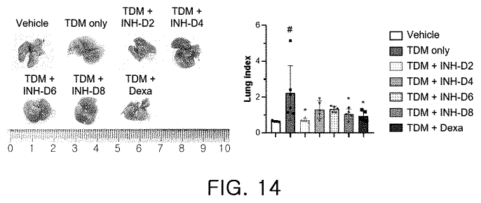

On day 7, mice were sacrificed, lungs were excised and weighed, and a lung index (lung weight/body weight×100) was calculated. The lung index was significantly decreased in the groups administered INH-D2 30 mg/kg, INH-D8 30 mg/kg, Dexa 0.5 mg/kg compared to the TDM only group. FIG. 14 shows photographs of lungs excised from mice sacrificed on day 7 (FIG. 14 (left)) and a graph of lung indices (lung weight/body weight×100) calculated after weighing for the lung granuloma induction group (TDM only) and for groups administered INH derivatives (INH-D2, INH-D4, INH-D6, and INH-D8) (FIG. 14 (right)).

### (2) Pulmonary Histopathological Analysis

The left lung lobes were fixed in 10% formalin for histopathological examination. Lung tissues were dehydrated, progressively immersed in alcohol and xylene, and embedded in paraffin. Tissue sections (5 μm) that were deparaffinized and stained with hematoxylin and eosin (H&E, #ab245880), at Abcam (Cambridge, UK) were used. Histopathological analysis of lung inflammation was performed using Motic Digital Slide Assistant software version 1.0.7.44 (Kowloon Bay, Kowloon, Hong Kong) (PMID: 31734231). High-resolution images of H&E-stained slides were scanned. Inflammation was measured in two steps using ImageJ software version 1.52 (NIH, Bethesda, MD; https://imagej.nih.gov/ij). The maximum threshold for all analyzed digital transparency within the lung area was set to 200. The minimum and maximum values of hue, saturation, and brightness of the images were set to 120, 255; 0, 255; and 0, 255, respectively. The total cell area was determined by modifying the procedure described in the online ImageJ Staining Examples directory (NIH, https://imagej.nih.gov/ij/docs/examples). The ratio of the area occupied by cells to the total area was used to calculate lung inflammation. Analysis showed that, compared with the TDM group, the INH-D2, INH-D8, and Dexa groups exhibited a significantly reduced lung granulomatous lesion index.

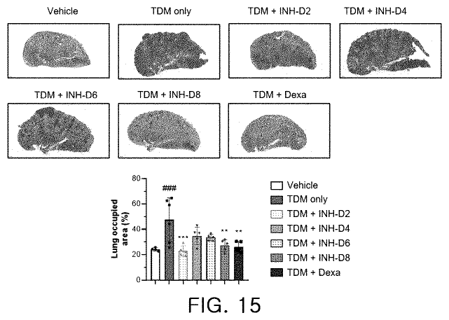

FIG. 15 shows, photographs of tissue lesions, FIG. 15 (upper panel), observed by histopathological analysis of lungs harvested from mice sacrificed on day 7 for the pulmonary granuloma induction group (TDM only) and for groups that administered INH derivatives (INH-D2, INH-D4, INH-D6, and INH-D8), and a graph of the area occupied by granulomatous lesions in the lungs, FIG. 15 (lower panel).

### 8. Statistical Analysis

Statistical comparisons were performed using a one-way analysis of variance (ANOVA). Significance levels were determined using Tukey's multiple comparison test in GraphPad Prism statistical software (version 8; San Diego, CA). P<0.05, P<0.01, and P<0.001 were considered statistically significant.

As a result of the above-described experiments, the isoniazid derivative of the present disclosure was confirmed to effectively suppress pulmonary granulomatous lesions and inflammatory mediators. By contrast, isoniazid, which is used as an therapeutic agent for tuberculosis, did not demonstrate reductions in inflammatory mediators or alleviation of pulmonary granulomatous lesions. In conclusion, the isoniazid derivative of the present disclosure may be used to alleviate pulmonary granulomatous lesions in terms of HDT during tuberculosis treatment.

While example embodiments have been shown and described above, it will be apparent to those skilled in the art that modifications and variations could be made without departing from the scope of the present invention as defined by the appended claims.

1. A compound represented by the following Chemical Formula 1 or a pharmaceutically acceptable salt thereof, for treating tuberculosis infections: 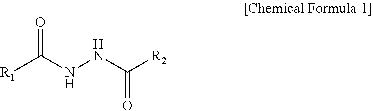
wherein, in Chemical Formula 1, R₁ and R₂ are independently selected from the group consisting of a nitrogen-containing C₃-C₉ heteroaryl group and a C₆-C₁₂ aryl group.

2. The compound or a pharmaceutically acceptable salt thereof according to claim 1, wherein R₁ is selected from the group consisting of pyridine, pyridazine, pyrimidine, and pyrazine, and R₂ is selected from the group consisting of pyridine, pyridazine, pyrimidine, pyrazine, and phenyl.

3. The compound or a pharmaceutically acceptable salt thereof according to claim 1, when R₁ is pyridine, the pyridine being bonded to the backbone at the 2- or 4-position, and when R₂ is pyridine, the pyridine being bonded to the backbone at the 3- or 4-position.

4. The compound or a pharmaceutically acceptable salt thereof according to claim 1, wherein the compound represented by Chemical Formula 1 is at least one compound selected from the group consisting of compounds represented by Chemical Formula D1 to D5:

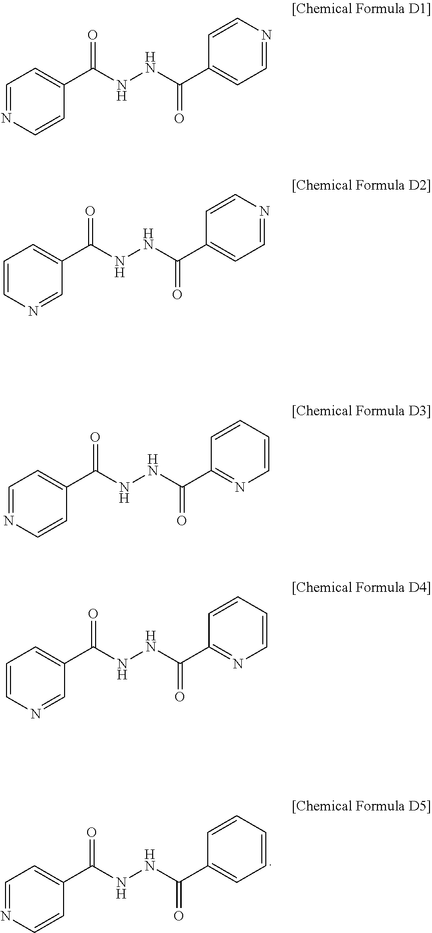 [SMILES: O=C(NNC(=O)c1ccncc1)c1ccncc1 ; O=C(NNC(=O)c1cccnc1)c1ccncc1 ; O=C(NNC(=O)c1ccccn1)c1ccncc1 ; O=C(NNC(=O)c1ccccn1)c1cccnc1 ; O=C(NNC(=O)c1ccncc1)c1ccccc1]

5. A pharmaceutical composition for the treatment of a disease caused by Mycobacterium tuberculosis infection, comprising as an active ingredient a compound represented by the following Chemical Formula 1 or a pharmaceutically acceptable salt thereof: 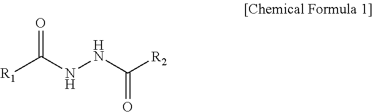
wherein in Chemical Formula 1, R₁ and R₂ are each independently selected from the group consisting of a nitrogen-containing C₃-C₉ heteroaryl group and a C₆-C₁₂ aryl group.

6. The pharmaceutical composition for the treatment of a disease caused by Mycobacterium tuberculosis infection according to claim 5, wherein R1 is selected from the group consisting of pyridine, pyridazine, pyrimidine, and pyrazine, and R2 is selected from the group consisting of pyridine, pyridazine, pyrimidine, pyrazine, and phenyl.

7. The pharmaceutical composition for the treatment of a disease caused by Mycobacterium tuberculosis infection according to claim 5, when R1 is pyridine, the pyridine being bonded to the backbone at the 2-position or the 4-position, and when R2 is pyridine, the pyridine being bonded to the backbone at the 3-position or the 4-position.

8. The pharmaceutical composition for the treatment of a disease caused by Mycobacterium tuberculosis infection according to claim 5, wherein the compound represented by Chemical Formula 1 is at least one selected from the group consisting of compounds of the following Chemical Formulae D1 to D5:

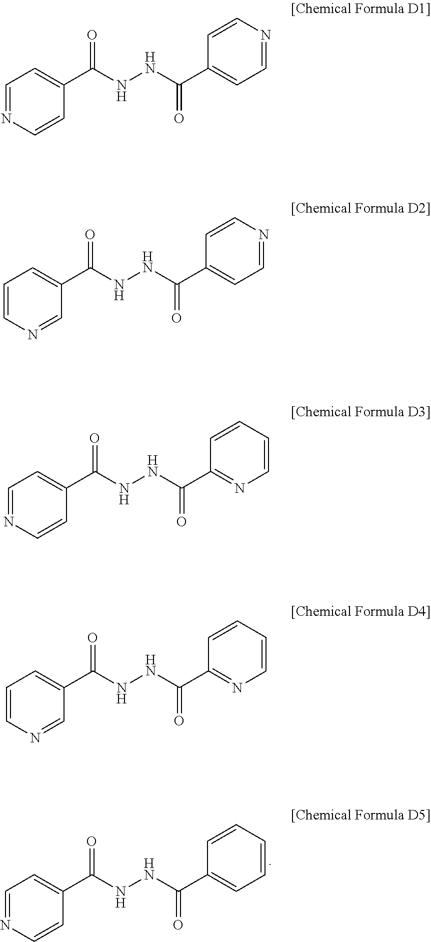 [SMILES: O=C(NNC(=O)c1ccncc1)c1ccncc1 ; O=C(NNC(=O)c1cccnc1)c1ccncc1 ; O=C(NNC(=O)c1ccccn1)c1ccncc1 ; O=C(NNC(=O)c1ccccn1)c1cccnc1 ; O=C(NNC(=O)c1ccncc1)c1ccccc1]

9. The pharmaceutical composition for the treatment of a disease caused by Mycobacterium tuberculosis infection according to claim 5, wherein the disease caused by Mycobacterium tuberculosis infection is at least one selected from the group consisting of ocular tuberculosis, cutaneous tuberculosis, renal tuberculosis, tuberculous lymphadenitis, laryngeal tuberculosis, intestinal tuberculosis, pulmonary tuberculosis, biliary tuberculosis, skeletal tuberculosis, pharyngeal tuberculosis, breast tuberculosis, and spinal tuberculosis.

10. The pharmaceutical composition for the treatment of a disease caused by Mycobacterium tuberculosis infection according to claim 5, wherein the disease caused by Mycobacterium tuberculosis infection is a granuloma.

11. The pharmaceutical composition for the treatment of a disease caused by Mycobacterium tuberculosis infection according to claim 5, wherein the tuberculosis is isoniazid-resistant tuberculosis (INHr-TB).

12. A method for treating tuberculosis infections, the method comprising administering to a subject in need thereof the compound according to claim 1 or the pharmaceutically acceptable salt thereof.

13. A method for treating a disease caused by Mycobacterium tuberculosis infection, the method comprising administering to a subject in need thereof the compound according to claim 1 or the pharmaceutically acceptable salt thereof.
